(19) United States
(12) Patent Application Publication
(10) Pub. No.: US 2025/0179007 A1
(43) Pub. Date: Jun. 5, 2025

(54) CERAMIDE COMPOUND CONTAINING CONJUGATED CARBOXYLIC ACID, PREPARATION METHOD THEREFOR AND APPLICATION

(71) Applicant: Shenzhen Dikeman Biotechnology Co., Ltd., Shenzhen (CN)

(72) Inventors: Chaowen Yang, Shenzhen (CN); Liu Ye, Shenzhen (CN)

(21) Appl. No.: 18/526,994

(22) Filed: Dec. 1, 2023

(51) Int. Cl. C07C 235/34; C07C 231/02; C07C 231/14; C07C 233/22; C07C 235/28; C07C 235/30

(52) U.S. Cl. CPC C07C 235/34; C07C 231/02; C07C 231/14; C07C 233/22; C07C 235/28; C07C 235/30

(57) ABSTRACT

The present invention provides A ceramide compound, comprising: a structure of general formula I or an enantiomer or diastereomer of general formula I:

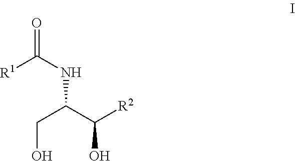

in which:
R¹ is selected from the condensation residues of ferulic acid, sinapic acid, abscisic acid, sotretinoin, tretinoin, fusidic acid, royal jelly acid, sorbic acid, caffeic acid or trans-cinnamic acid;
R² is selected from one of the following structures:—C₁₅H₂₉, —C₁₅H₃₁, —C₁₅H₂₇, —CHOHC₁₄H₂₇, —CHOHC₁₄H₂₉.

The present disclosure also provides a ceramide compound containing conjugated carboxylic acid, preparation method therefor and application thereof. The invention reacts functional carboxylic acid with sphingosine base to obtain a class of ceramide compound with novel structure. The introduction of physiologically active molecular fragments of functional carboxylic acids into ceramides will enhance the original efficacy of this type of molecules and also improve the physical and chemical properties of ceramide compound. For example, improving the antioxidant properties of ceramide compound.

### TECHNICAL FIELD

The present disclosure relates to a technical field of biomedicine, and in particular to a ceramide compound containing conjugated carboxylic acid, preparation method therefor and application thereof.

### BACKGROUND

Ceramide is one of the main components of the intercellular lipids that make up the stratum corneum of the skin. It is used to prevent excessive loss of water due to evaporation and maintain the structure of the stratum corneum. The stratum corneum acts as a barrier to protect underlying tissues from harmful substances or microorganisms from the external environment. When differentiated keratinocytes are shed from the stratum corneum, the intercellular lipids of the keratin form a lamellar structure that helps maintain the skin's basic functions. Intercellular lipids are composed of ceramide, cholesterol, free fatty acids and other components, and among these components, ceramide is known to play an important role in the water retention and barrier function of the stratum corneum. Reduced ceramide content in the stratum corneum is known to increase water evaporation and worsen various skin conditions. In addition, it is known that external supplementation of ceramides can restore skin to a normal state due to skin aging or external stimulation causing the ceramide content in the stratum corneum to be reduced. Therefore, research has been conducted on various natural animal and plant products containing ceramides. These ceramides, which exist in animals, plants, and microorganisms, are present in very small amounts, so there are problems with extraction difficulties and high production costs. In addition, natural ceramides have very low solubility in solvents commonly used in cosmetics, so it is difficult to make products contain an amount that can fully exert their effectiveness.

Due to the widespread demand for functional ceramides in the market, it is necessary to research and develop ceramide derivatives that can imitate natural ceramides and have enhanced efficacy. Such ceramides can solve the shortcomings of natural ceramides and can be produced commercially.

### SUMMARY

In order to solve the technical problem, the present disclosure provides a ceramide compound containing conjugated carboxylic acid, preparation method therefor and application thereof.

The object of the present invention is to provide a class of ceramide compound with novel structures, which contain conjugated carboxylic acids. Conjugated carboxylic acids refer to carboxylic acids in which a carboxyl group is conjugated to a carbon-carbon double bond. Carbon-carbon double bonds refer to straight-chain —CH═CH—, excluding ring formation such as cyclohexene, and carbon-carbon bonds of aromatic rings and heterocyclic rings.

The object of the present invention is to provide a synthesis method for ceramide compound.

Another object of the present invention is to provide the use of ceramide compound.

In order to achieve one of the above objects, the present invention adopts the following technical solutions:
a structure of general formula I or an enantiomer or diastereomer of general formula I:

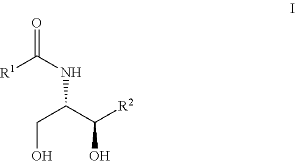

in which:
R¹ is selected from the condensation residues of ferulic acid, sinapic acid, abscisic acid, sotretinoin, tretinoin, fusidic acid, royal jelly acid, sorbic acid, caffeic acid or trans-cinnamic acid;
R² is selected from one of the following structures: —C₁₅H₂₉, —C₁₅H₃₁, —C₁₅H₂₇, —CHOHC₁₄H₂₇, —CHOHC₁₄H₂₉.

The residue after condensation refers to the remaining carboxylic acid fragment R after the carboxyl group of the corresponding carboxylic acid RCOOH condenses with the amino group of the sphingosine base to form a peptide bond. For example, the residue after acid condensation royal jelly is

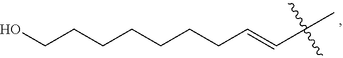 [SMILES: */C=C/CCCCCCCO]

the residue after condensation of ferulic acid is

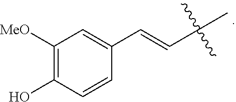 [SMILES: */C=C/c1ccc(O)c(OC)c1]

Among them, ferulic acid, as a phenolic plant component, has strong antioxidant activity and greatly promotes human health; ferulic acid can significantly increase the levels of glutathione and nicotinamide adenine dinucleotide phosphate in irradiated cells, and has a protective effect on irradiated endothelial cells.

Isotretinoin is suitable for severe acne, especially nodular cystic acne, and can also be used for diseases such as pityriasis rubra pilaris.

Tretinoin mainly affects bone growth and promotes epithelial cell proliferation, differentiation, keratolysis and other metabolic effects; tretinoin is used to treat acne vulgaris, psoriasis, ichthyosis, lichen planus, pityriasis rubra pilaris, keratosis pilaris, squamous cell carcinoma and melanoma.

Sorbic acid is a food additive and the most widely used preservative in the world. It has high antibacterial properties and can inhibit the growth and reproduction of mold.

Royal jelly acid, also known as 10-hydroxy-2-decenoic acid (10-HAD), is a component of royal jelly and is an unsaturated fatty acid. Royal jelly acid has the functions of enhancing the body's resistance, antibacterial and anti-inflammatory, and strongly inhibiting various cells such as lymphoma and breast cancer. It also has the functions of strengthening the body, preventing hair loss, and treating acute radiation damage and chemical substance damage.

Preferably, the R² is selected from one of the following structures:

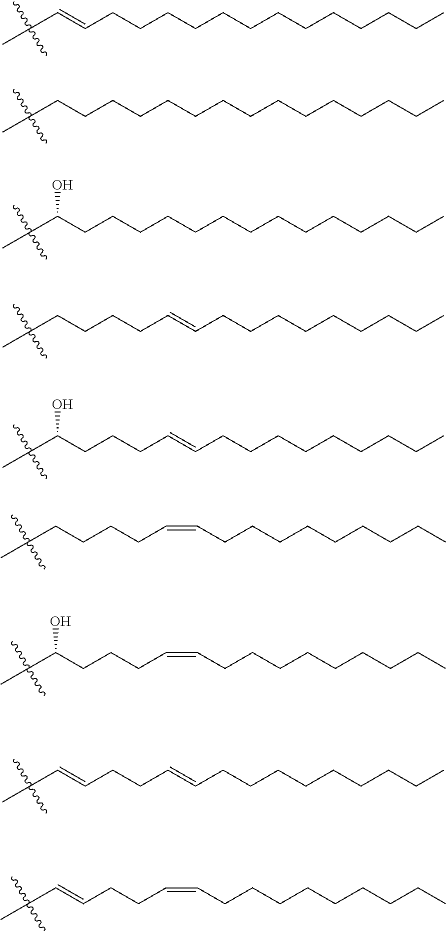

Further preferably, the R² is selected from one of the following structures:

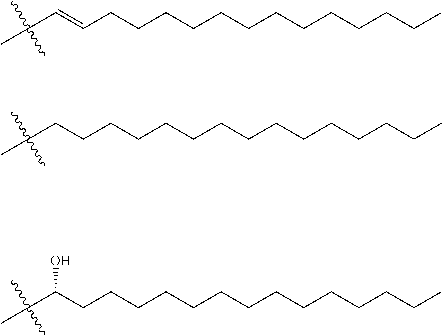 [SMILES: */C=C/CCCCCCCCCCCCC ; *CCCCCCCCCCCCCCC ; *[C@H](O)CCCCCCCCCCCCCC]

Corresponding to sphingosine, D-sphinganine, and phytosphingosine respectively.

Furthermore, the R¹ is selected from the condensation residues of ferulic acid, trans-cinnamic acid, royal jelly acid, sorbic acid, caffeic acid, tretinoin or sinapic acid.

Furthermore, the R¹ is selected from the condensation residues of ferulic acid, royal jelly acid, sorbic acid, caffeic acid, tretinoin or sinapic acid.

Furthermore, the R¹ is selected from the condensation residues of ferulic acid.

Furthermore, the R¹ is selected from the condensation residues of trans-cinnamic acid.

Furthermore, the R¹ is selected from the condensation residues of royal jelly acid.

Furthermore, the R¹ is selected from the condensation residues of sorbic acid.

Furthermore, the R¹ is selected from the condensation residues of caffeic acid.

Furthermore, the R¹ is selected from the condensation residues of tretinoin.

Furthermore, the R¹ is selected from the condensation residues of sinapic acid.

The ceramide compound is selected from one of the following compounds:

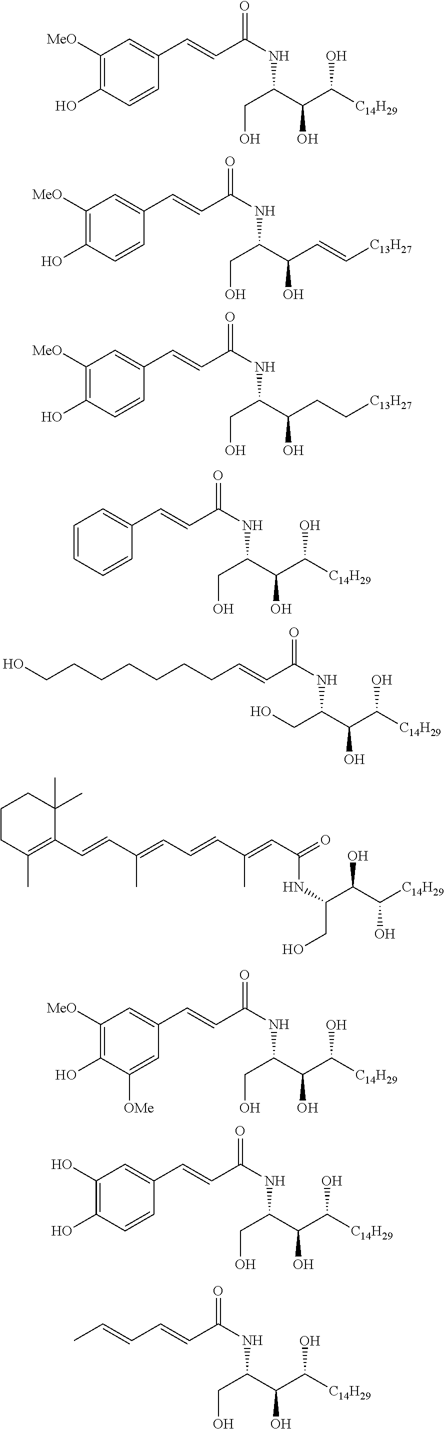

A preparation method of the ceramide compound of claim 5, wherein the following steps:

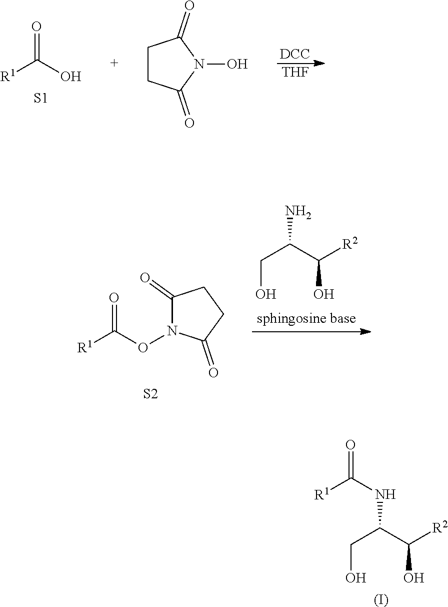

Compound S1 reacts with N-hydroxysuccinimide and a condensing agent to obtain compound S2.

Compound S2 reacts with sphingosine base to obtain compound I.

Furthermore, the molar ratio of compound S1, N-hydroxysuccinimide, the condensing agent, and sphingosine base is 1:(1˜2):(1˜2):(0.8˜1).

Furthermore, the condensation agent is DCC.

Furthermore, the solvent of the reaction is THF.

A preparation method of the ceramide compound of claim 1, wherein the following steps:

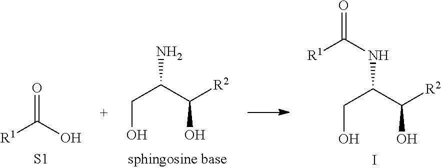

Compound S1 reacts with sphingosine base and the condensing agent to obtain compound I.

Furthermore, the condensing agent includes EDCI and HOBT; the molar ratio of compound S1, EDCI, HOBT, and sphingosine base is 1:(1˜2):(1˜2):(0.8˜1).

Furthermore, the condensation agent is DCC.

Furthermore, the solvent of the reaction is DCM.

The application of ceramide compound as antioxidants, especially in cosmetics, health products, and pharmaceuticals.

The invention has the following beneficial effects:

The invention reacts functional carboxylic acid with sphingosine base to obtain a class of ceramide compound with novel structure. The introduction of physiologically active molecular fragments of functional carboxylic acids into ceramides will enhance the original efficacy of this type of molecules and also improve the physical and chemical properties of ceramide compound. For example, improving the antioxidant properties of ceramide compound.

### BRIEF DESCRIPTION OF DRAWINGS

The invention will be further described with reference to the accompanying drawings, from which the object, technical solutions and beneficial effects of the invention will be clearer.

FIG. 1 is the test results of the antioxidant properties of the ceramide compound in Example 10.

### DETAILED DESCRIPTION

The present disclosure will be described in detail with reference to the following specific embodiments, which are used to help those skilled in the art to further understand the present disclosure, and shall not be construed to limit the present disclosure in any form. It would be appreciated by those skilled in the art that changes and modifications can be made in the embodiments without departing from the spirit of the present disclosure, which also belong to the protection scope of the present disclosure. It should be noted that the drawings provided in the following embodiments are only used to illustrate the basic idea of the present invention in a schematic manner, and the following embodiments and features in the embodiments can be combined with each other without conflict.

All reactions were performed under nitrogen atmosphere. Unless otherwise stated, chemicals were purchased commercially and without further purification. The dichloromethane and tetrahydrofuran used in the experiment are anhydrous solvents. Thin layer chromatography (TLC) uses 60F254 silica gel plates. Silica gel column chromatography uses Qingdao marine silica gel (particle size 0.040-0.063 mm). TLC color development uses UV light (254 nm) or iodine. The NMR spectrum was characterized using a Bruker DPX 400 nuclear magnetic resonance instrument. 1H NMR is 400 MHz. The solvent is Methanol-D4, deuterated DMSO or deuterated tetrahydrofuran, with tetramethylsilane (TMS) as the internal standard. The unit of chemical shift is ppm and the unit of coupling constant is Hz. In ¹H NMR, 8 represents a chemical shift, s represents a singlet, d represents a doublet, t represents a triplet, q represents a quartet, and m represents a multiplet.

DCC refers to dicyclohexylcarbodiimide, THF refers to tetrahydrofuran, DCM refers refers to dichloromethane, EDCI to N-(3-dimethylaminopropyl)-N′-ethyl-carbodiimide, HOBT Refers to 1-hydroxybenzotriazole.

General synthesis method of ceramide compound.

### Method A

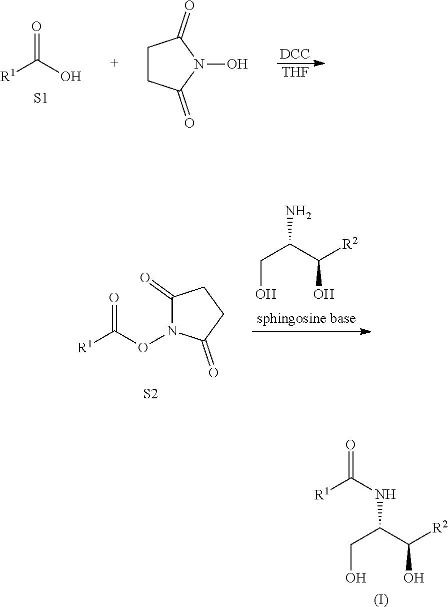

Step 1: place compound S1 (50 mmol), N-hydroxysuccinimide (60˜75 mmol, preferably 60 mmol), and DCC (60˜75 mmol, preferably 60 mmol) into a 250 ml round-bottomed flask, add 100 mL tetrahydrofuran, then stir at room temperature for 4 to 24 hours, and detect by TLC until compound S1 disappears completely. Post-treatment: filter the reaction system, collect the filtrate and use it directly for the next reaction.

step 2: add sphingosine base (40˜50 mmol, preferably 45 mmol) to the filtrate of the previous step, then stir at room temperature for 24˜72 hours, and detect by TLC until the sphingosine base completely disappears. Post-treatment: add saturated sodium bicarbonate aqueous solution to quench. The organic layer was separated, dried, filtered and concentrated in vacuo. The residue thus obtained was purified by silica gel column to obtain the product (50˜70% yield).

### Method B

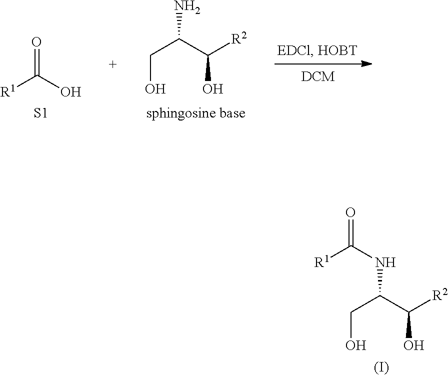

Place compound S1 (50 mmol), EDCI (60˜75 mmol, preferably 60 mmol), and HOBT (60˜75 mmol, preferably 60 mmol) into a 250 mL round-bottomed flask, add 100 mL DCM, then stir at room temperature for 1 hour, and then sphingosine base (40˜50 mmol, preferably 45 mmol) is added to the reaction system, and stirred at room temperature for 24˜72 hours until the sphingosine base disappears completely. Post-treatment: add water to quench. The organic layer was separated, dried, filtered and concentrated in vacuo. The residue thus obtained was purified by silica gel column to obtain the product (50˜65% yield).

### Example 1

The product of the condensation of ferulic acid and phytosphingosine by method A.

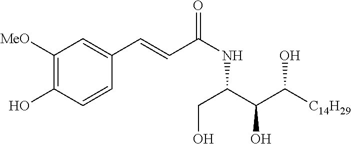

¹H NMR (400 MHZ, Methanol-d₄) δ 7.45 (d, J=15.6 Hz, 1H), 7.12 (d, J=2.0 Hz, 1H), 7.03 (dd, J=8.3, 1.9 Hz, 1H), 6.79 (d, J=8.1 Hz, 1H), 6.53 (d, J=15.7 Hz, 1H), 4.23 (q, J=5.3 Hz, 1H), 3.88 (s, 3H), 3.80 (qd, J=11.2, 5.0 Hz, 2H), 3.64 (t, J=5.8 Hz, 1H), 3.56 (ddd, J=8.9, 5.9, 2.5 Hz, 1H), 1.68 (dtd, J=12.9, 7.2, 6.2, 3.5 Hz, 1H), 1.53 (d, J=8.4 Hz, 1H), 1.43 (ddd, J=12.4, 8.1, 3.7 Hz, 1H), 1.36-1.16 (m, 23H), 0.89 (t, J=6.7 Hz, 3H).

### Example 2

The product of the condensation of ferulic acid and sphingosine base by method A.

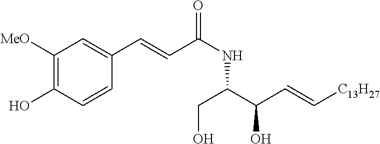

¹H NMR (400 MHZ, Methanol-d₄) δ 7.43 (d, J=15.7 Hz, 1H), 7.12 (d, J=2.0 Hz, 1H), 7.03 (dd, J=8.2, 2.0 Hz, 1H), 6.79 (d, J=8.1 Hz, 1H), 6.50 (d, J=15.7 Hz, 1H), 5.75-5.62 (m, 1H), 5.55-5.44 (m, 1H), 4.35 (t, J=7.0 Hz, 1H), 4.12 (t, J=7.4 Hz, 1H), 4.01-3.94 (m, 1H), 3.88 (s, 3H), 3.78-3.68 (m, 1H), 2.54-2.45 (m, 1H), 2.30-2.16 (m, 1H), 2.02 (p, J=7.2 Hz, 2H), 1.37-1.10 (m, 20H), 0.89 (t, J=6.9 Hz, 3H).

### Example 3

The product of the condensation of ferulic acid and D-sphinganine base by method A.

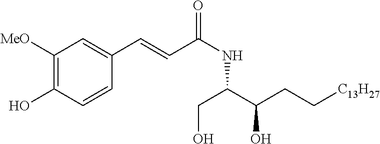

¹H NMR (400 MHZ, Methanol-d₄) δ 7.48 (d, J=15.7 Hz, 1H), 7.16 (d, J=2.0 Hz, 1H), 7.06 (dd, J=8.2, 1.9 Hz, 1H), 6.82 (d, J=8.2 Hz, 1H), 6.55 (d, J=15.7 Hz, 1H), 4.01-3.93 (m, 1H), 3.91 (s, 3H), 3.79 (d, J=5.0 Hz, 2H), 3.77-3.71 (m, 1H), 1.92-1.85 (m, 2H), 1.38-1.23 (m, 26H), 0.96-0.87 (m, 3H).

### Example 4

The product of the condensation of trans-cinnamic acid and phytosphingosine by method A.

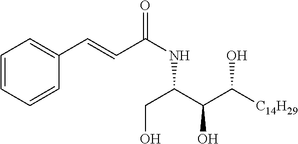

¹H NMR (400 MHZ, Methanol-d₄) δ 7.60-7.50 (m, 3H), 7.42-7.31 (m, 3H), 6.71 (d, J=15.8 Hz, 1H), 4.25 (td, J=5.7, 4.2 Hz, 1H), 3.83 (dd, J=11.3, 4.3 Hz, 1H), 3.77 (dd, J=11.3, 5.8 Hz, 1H), 3.64 (t, J=5.8 Hz, 1H), 3.56 (ddd, J=9.1, 5.9, 2.5 Hz, 1H), 1.75-1.61 (m, 1H), 1.53 (d, J=9.1 Hz, 1H), 1.48-1.16 (m, 24H), 0.90 (t, J=6.8 Hz, 3H).

### Example 5

The product of the condensation of royal jelly acid and phytosphingosine by method A.

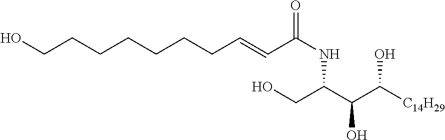

¹H NMR (600 MHZ, Methanol-d₄) δ 6.81 (dt, J=15.3, 7.0 Hz, 1H), 6.02 (dt, J=15.3, 1.5 Hz, 1H), 4.19 (td, J=5.8, 4.3 Hz, 1H), 3.81 (dd, J=11.3, 4.4 Hz, 1H), 3.76 (dd, J=11.2, 5.8 Hz, 1H), 3.62 (t, J=5.8 Hz, 1H), 3.58-3.53 (m, 3H), 2.23 (qd, J=7.2, 1.6 Hz, 2H), 1.57-1.49 (m, 5H), 1.39-1.29 (m, 32H), 0.92 (t, J=7.0 Hz, 3H).

### Example 6

The product of the condensation of tretinoin and phytosphingosine by method B.

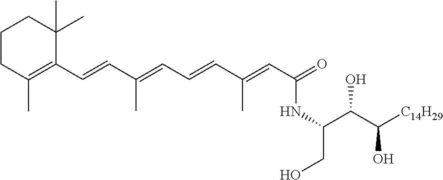

¹H NMR (400 MHZ, Methanol-d₄) δ 7.01 (dd, J=15.0, 11.4 Hz, 1H), 6.32 (dd, J=23.7, 15.6 Hz, 2H), 6.21-6.09 (m, 2H), 5.93 (s, 1H), 4.17 (td, J=5.7, 4.4 Hz, 1H), 3.86-3.72 (m, 2H), 3.64 (t, J=5.8 Hz, 1H), 3.61-3.53 (m, 1H), 2.33 (d, J=1.1 Hz, 3H), 2.06 (t, J=6.5 Hz, 2H), 2.01 (d, J=1.1 Hz, 3H), 1.73 (d, J=1.0 Hz, 3H), 1.66 (ddp, J=8.8, 5.6, 2.9 Hz, 3H), 1.54-1.47 (m, 3H), 1.30 (d, J=4.9 Hz, 25H), 1.05 (s, 6H), 0.92 (t, J=6.8 Hz, 3H).

### Example 7

The product of the condensation of sinapic acid and phytosphingosine by method B.

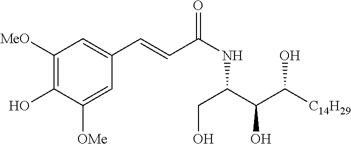

¹H NMR (400 MHZ, Methanol-d₄) δ 7.47 (d, J=15.6 Hz, 1H), 6.88 (s, 2H), 6.58 (d, J=15.6 Hz, 1H), 4.26 (td, J=5.6, 4.3 Hz, 1H), 3.89 (s, 6H), 3.85-3.78 (m, 2H), 3.67 (t, J=5.8 Hz, 1H), 3.59 (ddd, J=9.0, 5.9, 2.6 Hz, 1H), 1.77-1.61 (m, 2H), 1.61-1.43 (m, 2H), 1.29 (dd, J=11.8, 5.4 Hz, 22H), 0.92 (t, J=6.8 Hz, 3H).

### Example 8

The product of the condensation of caffeic acid and phytosphingosine by method B.

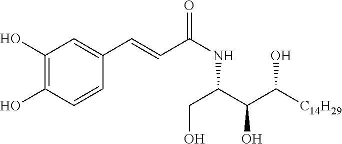

¹H NMR (400 MHZ, Methanol-d₄) δ 7.53 (d, J=15.9 Hz, 1H), 7.02 (d, J=2.1 Hz, 1H), 6.92 (dd, J=8.2, 2.1 Hz, 1H), 6.76 (d, J=8.2 Hz, 1H), 6.24 (d, J=15.9 Hz, 1H), 4.20 (q, J=5.3 Hz, 1H), 3.78 (qd, J=11.2, 5.0 Hz, 2H), 3.62 (t, J=6.0 Hz, 1H), 3.50 (ddd, J=8.9, 6.0, 2.4 Hz, 1H), 1.64 (dtd, J=12.9, 7.2, 6.2, 3.5 Hz, 1H), 1.51 (d, J=8.4 Hz, 1H), 1.43 (ddd, J=12.4, 8.1, 3.7 Hz, 1H), 1.36-1.16 (m, 23H), 0.89 (t, J=6.7 Hz, 3H).

### Example 9

The product of the condensation of sorbic acid and phytosphingosine by method B.

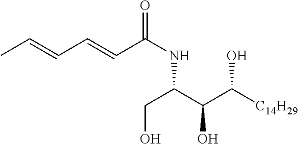

¹H NMR (400 MHZ, Methanol-d₄) δ 7.70 (td, J=6.8, 3.8 Hz, 1H), 7.32-7.23 (m, 1H), 7.14 (dd, J=15.1, 10.6 Hz, 1H), 6.25 (ddd, J=15.1, 10.6, 1.7 Hz, 1H), 6.13 (dq, J=15.2, 6.5 Hz, 1H), 6.00 (d, J=15.1 Hz, 1H), 4.20 (td, J=5.7, 4.3 Hz, 1H), 3.79 (qd, J=11.2, 5.1 Hz, 2H), 3.62 (t, J=5.8 Hz, 1H), 3.55 (ddd, J=8.9, 5.9, 2.5 Hz, 1H), 1.86 (dd, J=6.6, 1.4 Hz, 3H), 1.74-1.61 (m, 1H), 1.54 (d, J=9.0 Hz, 1H), 1.39-1.30 (m, 24H), 0.95-0.86 (m, 3H).

### Example 10

The antioxidant-ABTS method was used to test the antioxidant properties of the ceramide compound prepared in Examples 1, 2, 3, and 5.

Experimental principle: The use of ABTS to evaluate the antioxidant capacity of samples was first proposed by Miller et al. (1993). The method currently used is generally the method improved by Re et al. (1999). This method utilizes the fact that ABTS can be oxidized by a series of compounds such as potassium persulfate, hydrogen peroxide, and manganese dioxide to generate blue-green ABTS+ cationic free radicals with a maximum absorption peak at 734 nm. Under the action of antioxidants, ABTS+ is reduced to colorless ABTS. By measuring the absorbance value at 734 nm, the antioxidant capacity of the reactant can be determined.

ABTS free radical scavenging test antioxidant performance: Mix 3 mL ABTS aqueous solution (12 mmol/L) and 3 mL potassium persulfate solution (2.45 mmol/L) evenly, and keep it stable for 12 to 16 hours at room temperature in the dark. Use DMSO as the dilution medium, select an appropriate dilution ratio, and adjust the absorbance of the potassium persulfate solution at a wavelength of 734 nm to 0.700±0.025. Add samples of different concentrations and shake well, place in the dark for 10 minutes, and measure the absorbance value at 734 nm.

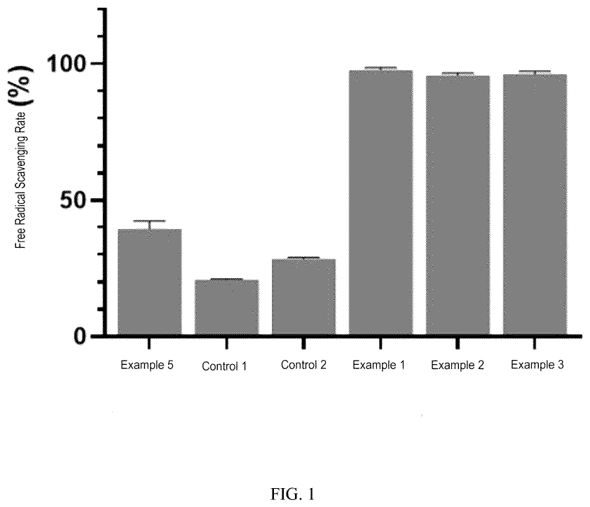

The results are shown in FIG. 1. Control 1 is Ceramide 3B, and Control 2 is Ceramide 3.

It can be seen that the compound of the present invention has a better antioxidant effect than the existing ceramide compound, and its free radical scavenging rate is generally 15 to 75% higher.

Above are only preferred embodiments of the present disclosure and are not intended to limit the present disclosure. Any modification, equivalent replacement and improvement made within spirit and principle of the present disclosure should be included in protective scope of the present disclosure.

What is claimed is:

1. A ceramide compound, comprising:
a structure of general formula I or an enantiomer or diastereomer of general formula I: 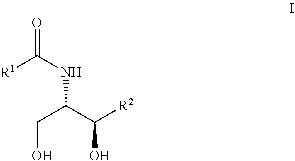
in which:
R¹ is selected from the condensation residues of ferulic acid, sinapic acid, abscisic acid, sotretinoin, tretinoin, fusidic acid, royal jelly acid, sorbic acid, caffeic acid or trans-cinnamic acid;
R² is selected from one of the following structures: —C₁₅H₂₉, —C₁₅H₃₁, —C₁₅H₂₇, —CHOHC₁₄H₂₇, —CHOHC₁₄H₂₉.

2. The ceramide compound of claim 1, wherein the R² is selected from one of the following structures:

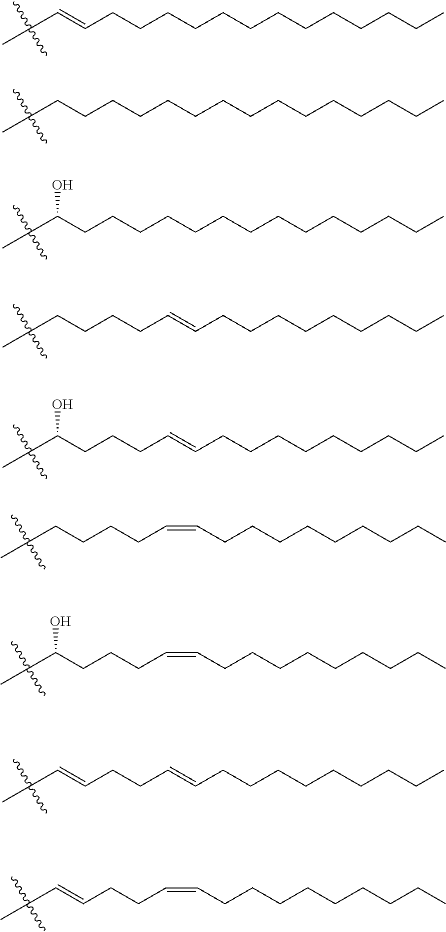

3. The ceramide compound of claim 2, wherein the R² is selected from one of the following structures:

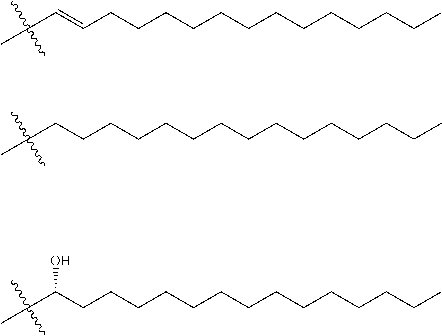 [SMILES: */C=C/CCCCCCCCCCCCC ; *CCCCCCCCCCCCCCC ; *[C@H](O)CCCCCCCCCCCCCC]

4. The ceramide compound of claim 1, wherein the R¹ is selected from the condensation residues of ferulic acid, trans-cinnamic acid, royal jelly acid, sorbic acid, caffeic acid, tretinoin or sinapic acid.

5. The ceramide compound of claim 1, wherein it is selected from one of the following compounds:

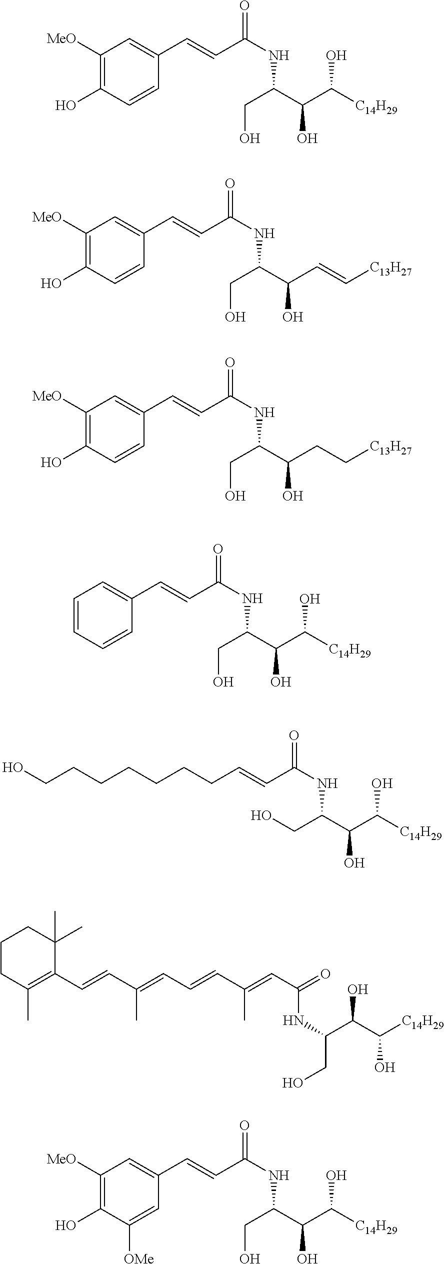

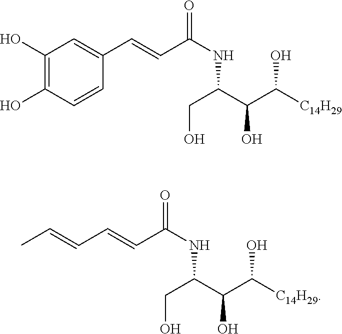

6. The application of the ceramide compound of claim 1 as antioxidants.

7. A preparation method of the ceramide compound, wherein the following steps: 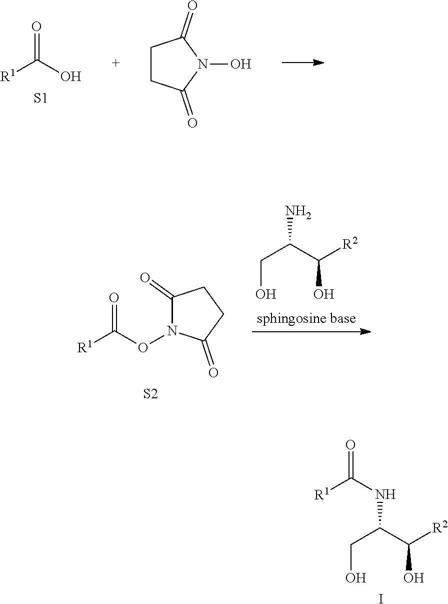
compound S1 reacts with N-hydroxysuccinimide and a condensing agent to obtain compound S2;
compound S2 reacts with sphingosine base to obtain compound I;
R¹ and R² are as defined in claim 1.

8. The preparation method of claim 7, wherein the molar ratio of compound S1, N-hydroxysuccinimide, the condensing agent, and sphingosine base is 1:(1˜2):(1˜2):(0.8˜1); the condensation agent is DCC; the solvent of the reaction is THF.

9. A preparation method of the ceramide compound, wherein the following steps: 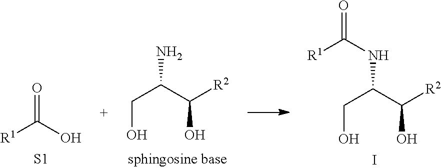
compound S1 reacts with sphingosine base and the condensing agent to obtain compound I;
R¹ and R² are as defined in claim 1.

10. The preparation method of claim 9, wherein the condensing agent includes EDCI and HOBT; the molar ratio of compound S1, EDCI, HOBT, and sphingosine base is 1:(1˜2):(1˜2):(0.8˜1); the condensation agent is DCC; the solvent of the reaction is DCM.
